(19) United States
(12) Patent Application Publication
(10) Pub. No.: US 2023/0135928 A1
(43) Pub. Date: May 4, 2023

(54) METHOD FOR TREATING LUNG DISEASE WITH CELL-FREE AMNIOTIC FLUID

(71) Applicant: UNIVERSITY OF UTAH RESEARCH FOUNDATION, Saly Lake City, UT (US)

(72) Inventors: Jan L. Pierce, Salt Lake City, UT (US); John D. Phillips, Salt Lake City, UT (US); Kevin T. Dustin, Salt Lake City, UT (US); Catherine Joy Lee, Salt Lake City, UT (US); Daniel Couriel, Salt Lake City, UT (US); Craig H. Selzman, Salt Lake City, UT (US); Giavonni M. Lewis, Salt Lake City, UT (US); Nathan Hatton, Salt Lake City, UT (US); Joseph Edward Tonna, Salt Lake City, UT (US)

(21) Appl. No.: 17/911,624

(22) PCT Filed: Apr. 2, 2021
(86) PCT No.: PCT/US2021/025494

§ 371 Date: Sep. 14, 2022

Related U.S. Application Data
(60) Provisional application No. 63/005,045, filed on Apr. 3, 2020.

(51) Int. Cl. A61K 35/50; A61K 47/36; A61K 9/19; A61K 9/00; A61P 11/00

(52) U.S. Cl. CPC A61K 35/50; A61K 47/36; A61K 9/19; A61K 9/0078; A61P 11/00

(57) ABSTRACT

The present disclosure relates to a cell-free sterile filtered amniotic fluid that may be processed and/or supplemented to treat lung inflammation and/or fibrosis caused by lung disease.

### CROSS-REFERENCE TO RELATED APPLICATIONS

This application claims priority to U.S. Provisional Patent Application No. 63/005,045, filed on Apr. 3, 2020, the contents of which are incorporated by reference in its entirety.

### TECHNICAL FIELD

Described herein is a cell-free sterile filtered amniotic fluid that may be processed and/or supplemented to treat lung inflammation and/or fibrosis caused by lung disease.

### BACKGROUND

The recent spread of COVID-19, the novel coronavirus, has been shown to increase inflammation in the body, especially the lungs. Interestingly, early research has shown that women who have been diagnosed with COVID-19 and give birth while infected do not transmit the virus to the baby. The virus was also not found in breast milk and the amniotic fluid (AF) that surrounds the baby.

AF is a protective fluid contained within the amniotic sac that surrounds a fetus during pregnancy. The AF can provide a number of developmental benefits to the fetus. For example, the AF can facilitate the exchange of proper nutritional and developmental components between the mother and the fetus to support proper tissue and organ development. However, the composition of AF typically changes over time. For example, during early stages of pregnancy the AF is often primarily an aqueous electrolyte solution. By about week 12 to week 14, AF begins to contain a variety of proteins, carbohydrates, lipids, urea, and the like.

AF is a rich source of nutrients, cytokines, chemokines, and growth factors that are valuable for fetal development and maturation. Additionally, AF also contains stem cells with the potential to differentiate along multiple cell lineages. Further, AF has a number of protective and regenerative properties, which can be provided via the exchange of water and solutes with surrounding tissues. This process can be accomplished via the utilization of different pathways during the course of a pregnancy that likely contribute to changes in the composition of the AF with gestational age.

Amniotic products, such as the fluid and the membrane, have been used to treat inflammation in other populations, such as people with burn injuries and tendon injuries, among other things. Use of amniotic products is shown to reduce inflammation.

Currently there is no treatment for COVID-19. An immediate treatment for COVID-19 is desperately needed to address the present pandemic. Lung inflammation and fibrosis caused by COVID-19 may lead to organ damage and ultimately death. Thus, there remains a need for a treatment for inflammation from respiratory system lung inflammation in subjects with lung diseases such as COVID-19.

### SUMMARY

One embodiment described herein is a method for treating lung disease in a subject in need thereof, the method comprising administering to the subject a therapeutically effective amount of a composition comprising a processed amniotic fluid having a therapeutically effective amount of protein, wherein the composition is substantially free of lanugo, vernix, and cells harvested with amniotic fluid. In one aspect, the protein comprises anti-inflammatory, anti-microbial, and/or immunomodulatory proteins. In another aspect, the lung disease comprises inflammation of the lungs due to asthma, bronchitis, chronic obstructive pulmonary disease (COPD), pneumonia, restrictive lung disease, bronchiectasis, pulmonary fibrosis, sarcoidosis, allergies, smoking, emphysema, acute respiratory distress syndrome (ARDS), interstitial lung disease (ILD), pneumoconiosis, lung cancer, or a combination thereof. In another aspect, the lung disease involves one or more of alveoli, trachea, interstitium, pluera, bronchi, and bronchioles. In another aspect, administration of the composition results in a reduction in lung inflammation or improves lung function. In another aspect, the lung disease is caused by a bacterial infection or a viral infection. In another aspect, the viral infection is caused by an influenza virus, a respiratory syncytial virus, an adenovirus, a Varicella zoster virus, or a coronavirus. In another aspect, the coronavirus comprises Severe Acute Respiratory Syndrome (SARS-CoV), Middle East Respiratory Syndrome (MERS-CoV), COVID-19 (2019-nCoV, SARS-CoV-2), 229E, NL63, OC43, or HKU1. In another aspect, the bacterial infection is caused by Streptococcus pneumoniae, Mycobacterium tuberculosis, Bordetella pertussis, Haemophilus influenzae, Moraxella catarrhalis, Pseudomonas aeruginosa, Stenotrophomonas maltophila, Staphylococcus aureus, Streptococcus pyogenes, Neisseria meningitidis, Klebsiella pneumoniae, or Nontuberculosis Mycobacterium. In another aspect, the symptoms of the lung disease include one or more of shortness of breath, wheezing, coughing, yellow mucosa, green mucosa, blood-tinged mucosa, chest pain, breathlessness, rapid breathing, hypoxia, inflammation of the lung tissue, or a decrease in blood pressure. In another aspect, the lung disease is acute or chronic. In another aspect, the composition is administrated to the subject in need thereof via one or more administration protocols comprising, intravenous injection or inhalation. In another aspect, the composition comprises a therapeutically effective amount of hyaluronic acid (HA). In another aspect, the therapeutically effective amount of protein is from about 0.15 mg/mL to about 10 mg/mL. In another aspect, the composition is lyophilized. In another aspect, the composition has an optical density of less than 0.20 when exposed to electromagnetic radiation at a wavelength of 590 nm in a liquid form. In another aspect, the composition has less than or equal to 10,000 particles per milliliter of particles having a particle size of 10 microns or greater. In another aspect, the composition has less than or equal to 300 particles per milliliter of particles having a particle size of 25 microns or greater. In another aspect, the composition further comprises an active agent. In another aspect, the active agent is an anti-infective agent, an antibiotic, an anti-tumor agent, an anti-inflammatory agent, a pain-controlling agent, an anti-rheumatic agent, a bisphosphonate, a supplementary growth factor, a supplementary cytokine, an amino acid, a protein, a vaccine, a hormone, a vitamin, a phytoestrogen, fluoride, or combinations thereof. In another aspect, the composition is administered via inhalation and/or intravenous injection. In another aspect, the composition is administered one or more times per day. In another aspect, the subject has been diagnosed with COVID-19. In another aspect, the composition is aerosolized and administered via a nebulizer. In another aspect, the subject receives at least 1 mL of the composition via inhalation every 24 hours. In another aspect, the subject further receives at least 1 mL of the composition intravenously every 24 hours.

Another embodiment described herein is the use of a therapeutically effective amount of a processed amniotic fluid having a therapeutically effective amount of protein, wherein the composition is substantially free of lanugo, vernix, and cells harvested with amniotic fluid for treating lung disease in a subject in need thereof.

### BRIEF DESCRIPTION OF THE DRAWINGS

FIG. 1 shows a schematic of the patient screening and enrolment flow chart.

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

As used herein, “formulation” and “composition” can be used interchangeably and refer to a combination of at least two ingredients. In some embodiments, at least one ingredient may be an active agent or otherwise have properties that exert physiologic activity when administered to a subject. For example, AF includes at least two ingredients (e.g., water and electrolytes) and is itself a composition or formulation.

As used herein, “therapeutic composition” and “pharmaceutical composition” can be used interchangeably and refer to a combination of at least two ingredients.

Given the successful applications of human amniotic products, and because respiratory inflammation is a core feature of chronic lung diseases and COVID-19, nebulizing cell-free amniotic fluid (AF) and/or delivering it intravenously may assist in the treatment of lung diseases and COVID-19, specifically by reducing inflammation in the lungs. In addition, COVID-19 causes an aggressive fibrotic response and, aside from mitigating inflammation, cell-free AF has been shown to decrease fibrosis. AF treatment may decrease the number of infected patients requiring critical care.

The inventors have demonstrated that rats undergoing myocardial ischemia repercussion that received either intramyocardial or intravenous injection of cell-free AF have a marked decrease in myocardial injury and fibrosis. Being that many of the end-stage COVID patients die of a myocarditis-type syndrome, systemic administration of cell-free AF may improve COVID-induced myocarditis-type syndrome as well.

Amniotic products (i.e., membrane and fluid) are FDA-approved as Human Cells, Tissues, and Cellular and Tissue-Based Products (HCT/Ps) regulated under 21 C.F.R. § 1271 for tissue healing and have been used as tissue transplants since 1910. In addition, there has never been a reported case of disease transmission associated with amniotic membrane after it has been secondarily sterilized using gamma irradiation. This evidence for amniotic membrane suggests that sterile filtered cell-free AF may be similarly safe.

Using cell-free AF as a treatment has proved safe for treatment of over 100 different diseases in tens of thousands of cases. However, its use for treatment of COVID-19 and inflammation of the respiratory system is unknown. Due to the beneficial combination of nutrients, cytokines, chemokines, growth factors, and the like that are present in AF, inflammation, and fibrosis in subjects with COVID-19 and/or inflammatory lung disease may be responsive to therapeutic treatment with AF.

### Amniotic Fluid

The term “amniotic fluid agent” refers to any protein, including but not limited to cytokines and chemokines, hyaluronic acid (HA), or other component typically found in AF to which an adverse health condition may be responsive and that is present in a therapeutic composition as described herein. In some examples, the “amniotic fluid agent” can be harvested with the AF of the therapeutic composition, can be supplemented into the therapeutic composition, or a combination thereof.

The therapeutic composition can include AF having a therapeutically effective amount of at least one protein, hyaluronic acid (HA), or a combination thereof. Further, the composition can be substantially free of lanugo, vernix, and cells harvested with or from the AF. The effective amount of at least one protein, HA, or both can be achieved through fortification or supplementation of the therapeutic composition with at least one protein, HA, or both.

The therapeutic composition can be made by extracting or harvesting an amount of AF from a pregnant female to provide an extracted or harvested AF. The harvested AF can include a therapeutically effective amount of at least one protein, hyaluronic acid, or both. The harvested AF can be centrifuged to form a supernatant and a cell pellet. A portion of the supernatant can be filtered to prepare the therapeutic composition, which can be substantially free of lanugo, vernix, and cells harvested with or from the AF.

AF can have antimicrobial, immunomodulatory, and growth-promoting properties. Components with antimicrobial, antiviral, and anti-fungal activity that are present in AF may include lysozyme, peroxidase, transferrin, β-lysin, immunoglobulins, zinc-peptide complexes, or combinations thereof. Moreover, AF can provide a variety of immunomodulatory properties, such as suppression of pro-inflammatory responses resulting from various adverse health conditions. Further, AF can provide a variety of growth promoting properties. As non-limiting examples, AF can enhance lung function by reducing lung damage, shortness of breath, wheezing, coughing, yellow mucus, green mucus, blood-tinged mucus, chest pain, breathlessness, rapid breathing, hypoxia, low blood pressure, or combinations thereof. Non-limiting examples of factors that are found in AF that can contribute to these activities can include inflammatory mediators such as cytokines that may include TNF-α, IL-6, IL-1ra, IL-1R4, Lactoferrin, Cystatin C, IL-8, IL-10, trophic factors that may include EGF, IGF-1, FGF, HGF and TGF-α, and HA.

The AF may be processed, which provides the AF with properties that are not present in freshly harvested AF. For example, the processed AF can have a reduced amount of particulate matter as compared to unprocessed AF. In some embodiments, the processed AF can have less than 10,000, less than 5000, less than 1000, less than 500, or less than 300 particles per mL of particles having a particle size of 10 microns or greater. In another embodiment, the processed AF can have less than 300, less than 200, less than 100, less than 50, or less than 30 particles per mL of particles having a particle size of 25 microns or greater.

Processed AF can also include a reduced amount of hemoglobin as compared to freshly harvested AF. For example, processed AF can include hemoglobin in an amount of from about 1 μg/mL to about 60 μg/mL, about 5 μg/mL to about 50 μg/mL, or about 10 μg/mL to about 40 μg/m L.

Processing of the amniotic fluid can also provide the amniotic fluid with a greater optical clarity (i.e., lower optical density) than freshly harvested amniotic fluid. For example, processed amniotic fluid can have an optical density of less than 0.20 when exposed to electromagnetic radiation at a wavelength of 590 nm, 570 nm, 550 nm, 540 nm, 500 nm, or 450 nm. In further examples, processed amniotic fluid can have an optical density of less than 0.15 when exposed to electromagnetic radiation at a wavelength of 590 nm, 570 nm, 550 nm, 540 nm, 500 nm, or 450 nm. In yet other examples, processed amniotic fluid can have an optical density of less than 0.10 when exposed to electromagnetic radiation at a wavelength of 590 nm, 570 nm, 550 nm, 540 nm, 500 nm, or 450 nm.

While processing can remove some constituents from the AF, a variety of beneficial constituents can also be largely preserved. For example, the processed AF can retain a comparable amount of total protein as found in freshly harvested AF. More specifically, total protein content for the AF composition can be within the range of about 0.15 mg/mL to about 10 mg/mL, about 0.5 mg/mL to about 5 mg/mL, about 1 mg/mL to about 3.0, about 1 mg/mL to about 3.5 mg/mL of total protein. Further, the processed AF can still include effective amounts of HA. For example, HA can be present in the AF in an amount greater than or equal to 150 ng/mL or from about 150 ng/mL to about 500 ng/mL, about 350 ng/mL to about 450 ng/mL, about 300 ng/mL to about 400 ng/mL, about 300 ng/mL to about 410 ng/mL, or about 300 ng/mL to about 420 ng/mL.

The AF may be lyophilized. The lyophilized AF can have a water content of from about 0.1 wt % to about 10 wt % prior to any desired subsequent dilution. The AF can also be concentrated by fortifying or supplementing the AF with at least one protein, HA, or both, as desired for a particular application of the therapeutic composition. For example, the therapeutic composition can include only processed AF, which can be diluted and/or concentrated as desired. In other examples, the therapeutic composition can be fortified or supplemented with at least one protein that can be naturally found in AF, such as HA, a cytokine, a growth factor, stem cells, nutrients, electrolytes, etc.

### Pharmaceutical Compositions

The disclosed AF compositions may be incorporated into pharmaceutical compositions suitable for administration to a subject (such as a patient, which may be a human or non-human). The pharmaceutical composition may be prepared for administration to a subject. Such pharmaceutical compositions can be administered in dosages and by techniques well known to those skilled in the medical and pharmaceutical arts taking into consideration such factors as the age, sex, weight, and condition of the particular subject, and the route of administration.

The pharmaceutical compositions and formulations may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of AF of the disclosure are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

For example, a therapeutically effective amount of AF may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The pharmaceutical compositions and formulations may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the AF and components thereof and their physiologically acceptable salts may be formulated for administration by, for example, injection, inhalation (either through the mouth or the nose), solid dosing, eye drop, in a topical oil-based formulation, implants, oral, buccal, parenteral, or rectal administration. Techniques and formulations may generally be found in “Remington's Pharmaceutical Sciences,” (Meade Publishing Co., Easton, Pa.). Therapeutic compositions must typically be sterile and stable under the conditions of manufacture and storage.

The route by which the disclosed AF compositions are administered, and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50% to about 90%, about 50% to about 80%, about 50% to about 70%, about 50% to about 60%, about 60% to about 90%, about 60% to about 80%, about 60% to about 70%, about 70% to about 90%, about 70% to about 80%, or about 80% to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5% to about 10%, about 5% to about 9%, about 5% to about 8%, about 5% to about 7%, about 5% to about 6%, about 6% to about 10%, about 6% to about 9%, about 6% to about 8%, about 6% to about 7%, about 7% to about 10%, about 7% to about 9%, about 7% to about 8%, about 8% to about 10%, about 8% to about 9%, or about 9% to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5% to about 50%, about 5% to about 45%, about 5% to about 40%, about 5% to about 35%, about 5% to about 30%, about 5% to about 25%, about 5% to about 20%, about 5% to about 15%, about 5% to about 10%, about 10% to about 50%, about 10% to about 45%, about 10% to about 40%, about 10% to about 35%, about 10% to about 30%, about 10% to about 25%, about 10% to about 20%, about 10% to about 15%, about 15% to about 50%, about 15% to about 45%, about 15% to about 40%, about 15% to about 35%, about 15% to about 30%, about 15% to about 25%, about 15% to about 20%, about 20% to about 50%, about 20% to about 45%, about 20% to about 40%, about 20% to about 35%, about 20% to about 30%, about 20% to about 25%, about 25% to about 50%, about 25% to about 45%, about 25% to about 40%, about 25% to about 35%, about 25% to about 30%, about 30% to about 50%, about 30% to about 45%, about 30% to about 40%, about 30% to about 35%, about 35% to about 50%, about 35% to about 45%, about 35% to about 40%, about 40% to about 50%, about 40% to about 45%, or about 45% to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1% to about 10.0%, about 0.5% to about 10.0%, about 1.0% to about 10.0%, about 1.5% to about 10.0%, about 2.0% to about 10.0%, about 2.5% to about 10.0%, about 3.0% to about 10.0%, about 3.5% to about 10.0%, about 4.0% to about 10.0%, about 4.5% to about 10.0%, about 5.0% to about 10.0%, about 5.5% to about 10.0%, about 6.0% to about 10.0%, about 6.5% to about 10.0%, about 7.0% to about 10.0%, about 7.5% to about 10.0%, about 8.0% to about 10.0%, about 8.5% to about 10.0%, about 9.0% to about 10.0%, or about 9.5% to about 10.0%.

Suitable colorants include a colorant such as an FD&C dye. When used, the amount of colorant in a systemic or topical composition is typically about 0.005% to about 0.1%, about 0.010% to about 0.1%, about 0.015% to about 0.1%, about 0.020% to about 0.1%, about 0.025% to about 0.1%, about 0.030% to about 0.1%, about 0.035% to about 0.1%, about 0.040% to about 0.1%, about 0.045% to about 0.1%, about 0.050% to about 0.1%, about 0.055% to about 0.1%, about 0.060% to about 0.1%, about 0.065% to about 0.1%, about 0.070% to about 0.1%, about 0.075% to about 0.1%, about 0.080% to about 0.1%, about 0.085% to about 0.1%, about 0.090% to about 0.1%, or about 0.095% to about 0.1%.

Suitable flavors include menthol, peppermint, and fruit flavors. The amount of flavor(s), when used, in a systemic or topical composition is typically about 0.1% to about 1.0%, about 0.2% to about 1.0%, about 0.3% to about 1.0%, about 0.4% to about 1.0%, about 0.5% to about 1.0%, about 0.6% to about 1.0%, about 0.7% to about 1.0%, about 0.8% to about 1.0%, or about 0.9% to about 1.0%.

Suitable sweeteners include aspartame and saccharin. The amount of sweetener(s) in a systemic or topical composition is typically about 0.001% to about 1.0%, about 0.005% to about 1.0%, about 0.010% to about 1.0%, about 0.015% to about 1.0%, about 0.020% to about 1.0%, about 0.025% to about 1.0%, about 0.030% to about 1.0%, about 0.035% to about 1.0%, about 0.040% to about 1.0%, about 0.045% to about 1.0%, about 0.050% to about 1.0%, about 0.055% to about 1.0%, about 0.060% to about 1.0%, about 0.065% to about 1.0%, about 0.070% to about 1.0%, about 0.075% to about 1.0%, about 0.080% to about 1.0%, about 0.085% to about 1.0%, about 0.090% to about 1.0%, or about 0.095% to about 1.0%.

Suitable antioxidants include butylated hydroxyanisole (“BHA”), butylated hydroxytoluene (“BHT”), and vitamin E. The amount of antioxidant(s) in a systemic or topical composition is typically about 0.1% to about 5.0%, about 0.5% to about 5.0%, about 1.0% to about 5.0%, about 1.5% to about 5.0%, about 2.0% to about 5.0%, about 2.5% to about 5.0%, about 3.0% to about 5.0%, about 3.5% to about 5.0%, about 4.0% to about 5.0%, or about 4.5% to about 5.0%.

Suitable preservatives include benzalkonium chloride, methyl paraben and sodium benzoate. The amount of preservative(s) in a systemic or topical composition is typically about 0.01% to about 5.0%, about 0.10% to about 5.0%, about 0.20% to about 5.0%, about 0.30% to about 5.0%, about 0.40% to about 5.0%, about 0.50% to about 5.0%, about 0.60% to about 5.0%, about 0.70% to about 5.0%, about 0.80% to about 5.0%, about 0.90% to about 5.0%, about 1.00% to about 5.0%, about 1.10% to about 5.0%, about 1.20% to about 5.0%, about 1.30% to about 5.0%, about 1.40% to about 5.0%, about 1.50% to about 5.0%, about 1.60% to about 5.0%, about 1.70% to about 5.0%, about 1.80% to about 5.0%, about 1.90% to about 5.0%, about 2.00% to about 5.0%, about 2.10% to about 5.0%, about 2.20% to about 5.0%, about 2.30% to about 5.0%, about 2.40% to about 5.0%, about 2.50% to about 5.0%, about 2.60% to about 5.0%, about 2.70% to about 5.0%, about 2.80% to about 5.0%, about 2.90% to about 5.0%, about 3.00% to about 5.0%, about 3.10% to about 5.0%, about 3.20% to about 5.0%, about 3.30% to about 5.0%, about 3.40% to about 5.0%, about 3.50% to about 5.0%, about 3.60% to about 5.0%, about 3.70% to about 5.0%, about 3.80% to about 5.0%, about 3.90% to about 5.0%, about 4.00% to about 5.0%, about 4.10% to about 5.0%, about 4.20% to about 5.0%, about 4.30% to about 5.0%, about 4.40% to about 5.0%, about 4.50% to about 5.0%, about 4.60% to about 5.0%, about 4.70% to about 5.0%, about 4.80% to about 5.0%, or about 4.90% to about 5.0%.

Suitable glidants include silicon dioxide. The amount of glidant(s) in a systemic or topical composition is typically about 1% to about 5%, about 2% to about 5%, about 3% to about 5%, or about 4% to about 5%.

Suitable solvents include water, isotonic saline, ethyl oleate, glycerin, hydroxylated castor oils, alcohols such as ethanol, and phosphate buffer solutions. The amount of solvent(s) in a systemic or topical composition is typically from about 0% to about 100%, about 5% to about 100%, about 10% to about 100%, about 15% to about 100%, about 20% to about 100%, about 25% to about 100%, about 30% to about 100%, about 35% to about 100%, about 40% to about 100%, about 45% to about 100%, about 50% to about 100%, about 55% to about 100%, about 60% to about 100%, about 65% to about 100%, about 70% to about 100%, about 75% to about 100%, about 80% to about 100%, about 85% to about 100%, about 90% to about 100%, or about 95% to about 100%.

Suitable suspending agents include AVICEL RC-591 (from FMC Corporation of Philadelphia, Pa.) and sodium alginate. The amount of suspending agent(s) in a systemic or topical composition is typically about 1% to about 8%, about 2% to about 8%, about 3% to about 8%, about 4% to about 8%, about 5% to about 8%, about 6% to about 8%, or about 7% to about 8%.

Suitable surfactants include lecithin, sodium lauryl sulfate, and polyethylene glycol sorbitan monolaurates (Tween®) (Croda International). Suitable surfactants include those disclosed in the C.T.F.A. Cosmetic Ingredient Handbook, 1992, pp. 587-592; Remington's Pharmaceutical Sciences, 15th ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5.0%, about 0.5% to about 5.0%, about 1.0% to about 5.0%, about 1.5% to about 5.0%, about 2.0% to about 5.0%, about 2.5% to about 5.0%, about 3.0% to about 5.0%, about 3.5% to about 5.0%, about 4.0% to about 5.0%, or about 4.5% to about 5.0%.

Although the amounts of components in the systemic compositions may vary depending on the type of systemic composition prepared, in general, systemic compositions include 0.01% to 50% of an active compound (e.g., AF) and 50% to 99.99% of one or more carriers. Compositions for parenteral administration typically include 0.1% to 10% of actives and 90% to 99.9% of a carrier including a diluent and a solvent.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include AF and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of AF include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The amount of the carrier employed in conjunction with AF as disclosed herein is sufficient to provide a practical quantity of composition for administration per unit dose of AF. Techniques and compositions for making dosage forms useful in the methods of this disclosure are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2^{nd} ed., (1976).

The therapeutic composition can include an active agent that is not typically found in AF. For example, the therapeutic composition can include a variety of additives and active agents such as an anti-infective agent, an anti-inflammatory agent, a pain-controlling agent, a supplementary cytokine, a chemokine, an amino acid, a protein, a vaccine, a hormone, a vitamin, pH adjusting additive, the like, or combinations thereof.

Anti-infective agents can be a variety of active agents that can kill or prevent an infectious organism from spreading. Thus, anti-infective agents can include antibacterial agents, antifungal agents, antiviral agents, anti-protozoan agents, the like, or combinations thereof. Non-limiting examples can include amebicides such as chloroquine, nitazoxanide, paromomycin, tinidazole, metronidazole, iodoquinole, or the like; aminoglycosides such as tobramycin, gentamicin, amikacin, kanamycin, neomycin, streptomycin, or the like; anthelmintics such as albendazole, ivermectin, praziquantel, pyrantel, mebendazole, miltefosine, niclosamide, piperazine, thiabendazole, or the like; antifungals such as itraconazole, posaconazole, ketoconazole, fluconazole, clotrimazole, isavuconazole, miconazole, voriconazole, echinocandins, terbinafine, griseofulvin, flucytosine, nystatin, amphotericin b, or the like; antimalarials such as chloroquine, quinine, hydroxychloroquine, mefloquine, primaquine, pyrimethamine, halofantrine, doxycycline, or the like; antituberculosis agents such as aminosalicylic acid, bedaquiline, isoniazid, ethambutol, pyrazinamide, ethionamide, rifampin, rifabutin, rifapentine, capreomycin, cycloserine, streptomycin, or the like; antivirals such as amantadine, rimantadine, ritonavir, cobicistat, peginterferon alfa 2a, peginterferon alfa 2b, maraviroc, raltegravir, dolutegravir, elvitegravir, sofosbuvir, enfuvirtide, fomivirsen, foscarnet, oseltamivir, zananivir, peramivir, etravirine, efavirenz, nevirapine, delavirdine, rilpivirine, daclatasvir, adefovir, entecavir, telbivudine, didanosine, tenofovir, abacavir, lamivudine, zidovudine, stavudine, emtricitabine, zalcitabine, boceprevir, simeprevir, fosamprenavir, lopinavir, darunavir, telaprevir, ritonavir, tipranavir, atazanavir, nelfinavir, amprenavir, indinavir, saquinavir, ganciclovir, valacyclovir, famciclovir, acyclovir, valganciclovir, ribavirin, cidofovir, or the like; carbapenems such as doripenem, meropenem, cilastatin, ertapenem, or the like; cephalosporins such as avibactam, ceftolozane, ceftazidime, tazobactam, cefadroxil, cephalexin, cefazolin, ceftaroline, loracarbef, cefotetan, cefuroxime, cefprozil, cefaclor, cefoxitin, ceftibuten, cefotaxime, ceftriaxone, cefpodoxime, cefixime, cefdinir, defditoren, ceftazidime, ceftizoxime, cefepime, or the like; glycopeptide antibiotics such as vancomycin, dalbavancin, oritavancin, telavancin, or the like; glycocyclines such as tigecycline, or the like; leprostatics such as thalidomide, dapsone, clofazimine, or the like; lincomycin, or the like; clindamycin, or the like; ketolides such as telithromycin, or the like; macrolides such as azithromycin, fidaxomicin, erythromycin, clarithromycin, or the like; antibiotics such as aztreonam, daptomycin, chloramphenicol, colistimethate, fosfomycin, rifaximin, metronidazole, sulfamethoxazole, atovaquone, bacitracin, dalfopristin, erythromycin, furazolidone, pentamidine, polymyxin b, spectinomycin, trimetrexate, linezolid, tedizolid, penicillins (e.g., ampicillin, amoxicillin, carbenicillin, piperacillin, ticarcillin, nafcillin, dicloxacillin, cloxacillin, oxacillin, or the like), quinolones (e.g., lomefloxacin, norfloxa-cin, ofloxacin, gatifloxacin, moxifloxacin, ciprofloxacin, levofloxacin, gemifloxacin, cinoxacin, nalidixic acid, spar-floxacin, or the like), sulfonamides (e.g., sulfamethoxazole, sulfadiazine, sulfisoxazole, or the like), tetracyclines (e.g., tetracycline, demeclocycline, doxycycline, minocycline, or the like), or the like; urinary anti-infectives such as methenamine, methylene blue, fosfomycin, nitrofurantoin, trimethoprim, cinoxacin, nalidixic acid, oxytetracycline, or the like; hydrates thereof, acids thereof, bases thereof, salts thereof, or combinations of any of such anti-infective agents.

Non-limiting examples of anti-inflammatory agents can include ibuprofen, naproxen, aspirin, diclofenac, celecoxib, sulindac, oxaprozin, piroxicam, indomethacin, meloxicam, fenoprofen, difunisal, etodolac, ketorolac, meclofenamate, nabumetone, salsalate, ketoprofen, tolmetin, flurbiprofen, mefenamic acid, famotidine, bromfenac, nepafenac, prednisone, cortisone, hydrocortisone, methylprednisolone, deflazacort, prednisolone, fludrocortisone, amcinonide, betamethasone diproprionate, clobetasol, clocortolone, dexamethasone, diflorasone, dutasteride, flumethasone pivalate, flunisolide, fluocinolone acetonide, fluocinonide, fluorometholone, fluticasone propionate, flurandrenolide, hydroflumethiazide, the like, hydrates thereof, acids thereof, bases thereof, or salts thereof, or combinations thereof.

Non-limiting examples of pain controlling agents can include anti-inflammatory agents, such as those listed above, acetaminophen, codeine, dihydrocodeine, tramadol, meperidine, hydrocodone, oxycodone, morphine, fentanyl, hydromorphone, buprenorphine, methadone, diamorphine, pethidine, the like, hydrates thereof, acids thereof, bases thereof, or salts thereof, or combinations thereof.

Non-limiting examples of vaccines can include adenovirus vaccine, coxsackie B vaccine, cytomegalovirus vaccine, dengue vaccine, Eastern equine encephalitis vaccine, Ebola vaccine, enterovirus vaccine, Epstein-barr vaccine, hepatitis A vaccine, hepatitis B vaccine, hepatitis C vaccine, hepatitis E vaccine, HIV vaccine, human papillomavirus vaccine, HTLV-1 T-lymphotrophic vaccine, influenza vaccine, Japanese encephalitis vaccine, Marburg vaccine, measles vaccine, mumps vaccine, norovirus vaccine, polio vaccine, rabies vaccine, respiratory syncytial virus (RSV) vaccine, rotavirus vaccine, rubella vaccine, severe acute respiratory syndrome (SARS) vaccine, varicella vaccine, smallpox vaccine, West Nile virus vaccine, yellow fever vaccine, anthrax vaccine, DPT vaccine, Q fever vaccine, Hib vaccine, tuberculosis vaccine, meningococcal vaccine, typhoid vaccine, pneumococcal vaccine, cholera vaccine, caries vaccine, ehrlichiosis vaccine, leprosy vaccine, Lyme disease vaccine, Staphylococcus aureus vaccine, Streptococcus pyogenes vaccine, syphilis vaccine, tularemia vaccine, Yersinia pestis vaccine, the like, or combinations thereof.

Non-limiting vitamins can include vitamin A, vitamin B1, vitamin B2, vitamin B6, vitamin B12, vitamin C, vitamin D, vitamin E, vitamin H, vitamin K, folic acid, the like, or combinations thereof.

Suitable pH adjusting additives may include HCl or NaOH in amounts sufficient to adjust the pH of the therapeutic composition.

### Pharmaceutically Acceptable Salts

AF and components thereof as disclosed herein may exist as a pharmaceutically acceptable salt. The term “pharmaceutically acceptable salt” refers to salts or zwitterions of AF and components thereof which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the AF and components thereof or separately by reacting an amino group of the AF and components thereof with a suitable acid. For example, AF and components thereof may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthalenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the AF and components thereof may also be quaternized with alkyl chlorides, bromides, and iodides such as methyl, ethyl, propyl, isopropyl, butyl, lauryl, myristyl, stearyl, and the like.

Basic addition salts may be prepared during the final isolation and purification of the disclosed AF and components thereof by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### Methods of Treatment

The AF and components thereof and pharmaceutical compositions described herein may be useful for treating the disorders described herein in a subject in need thereof.

As used herein, the terms “subject” and “patient” may be used interchangeably to refer to any vertebrate including, but not limited to, a mammal and a human. In some embodiments, the subject may be a human or a non-human. The subject or patient may be undergoing forms of treatment.

As used herein, the terms “treat,” “treating” or “treatment” are each used interchangeably to describe reversing, alleviating, or inhibiting the progress of a disease and/or injury, or one or more symptoms of such disease, to which such term applies. Depending on the condition of the subject, the term may also refer to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a pharmaceutical composition to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

The term “dosage unit” or “dose” are understood to mean an amount of an active agent that is suitable for administration to a subject in order achieve or otherwise contribute to a therapeutic effect. In some examples, a dosage unit can refer to a single dose that is capable of being administered to a subject or patient, and that may be readily handled and packed, remaining as a physically and chemically stable unit dose.

For example, administration of the composition to the subject results in inhibition or slowing of the lung disease.

In another example, administration of the composition to the subject may result in inhibition or slowing of the normal rate of increase of viral load as compared to an untreated subject. As used herein, the term “viral load” is a measurement of the amount of a virus in a subject.

The lung inflammation may be associated with, but not limited to respiratory failure, respiratory distress, pulmonary disease, cystic fibrosis, asthma, bronchitis, inflammation/swelling of the lungs, chronic obstructive pulmonary disease (COPD), pneumonia, restrictive lung disease, bronchiectasis, pulmonary fibrosis, sarcoidosis, allergies, smoking, emphysema, acute respiratory distress syndrome (ARDS), interstitial lung disease (ILD), pneumoconiosis or lung cancer. The lung diseases may affect the alveoli, trachea, interstitium, pluera, bronchi and/or bronchioles. The lung disease may cause diffuse alveolar damage, denuded alveolar lining cells with reactive type II pneumocyte hyperplasia, intra-alveolar fibrinous exudates, loose interstitial fibrosis, intra-alveolar loose fibrous plugs of organizing pneumonia, intra-alveolar organizing fibrin, damaged alveolar epithelial cells, desquamated cells within the alveolar space, cellular fibromyxoid exudates, desquamation of pneumocytes, hyaline membrane formation (e.g., indication of ARDS), pulmonary oedema, (e.g., early-phase ARDS). The lung disease may also cause chronic inflammation such as interstitial mononuclear inflammatory infiltrates dominated by lymphocytes. The lung disease may cause infiltration of the intra-alveolar spaces in the lung by multinucleated syncytial cells with atypical enlarged pneumocytes characterized by large nuclei, amphophilic granular cytoplasm, and/or prominent nucleoli that show viral cytopathic-like changes. The lung disease may cause increased inflammatory FCN1+ macrophages that replace FABP4+ macrophages in severe disease. The lung disease may cause highly expanded and functional competent tissue resident clonal CD8+ T cells in mild disease. Blood vessels or interstitial areas between alveoli may not be affected by the lung disease.

The lung disease may be caused by a bacterial infection or a viral infection. The bacterial infection may be caused by Streptococcus pneumoniae, Mycobacterium tuberculosis, Bordetella pertussis, Haemophilus influenzae, Moraxella catarrhalis, Pseudomonas aeruginosa, Stenotrophomonas maltophila, Staphylococcus aureus, Streptococcus pyogenes, Neisseria meningitidis, Klebsiella pneumoniae, or Nontuberculosis Mycobacterium. The viral infection may be caused by an influenza virus, a respiratory syncytial virus, an adenovirus, a Varicella zoster virus, or a coronavirus. The coronavirus may be Severe Acute Respiratory Syndrome (SARS-CoV), Middle East Respiratory Syndrome (MERS-CoV), COVID-19 (2019-nCoV, SARS-CoV-2), 229E, NL63, 0043, or HKU1. In a particular embodiment, the coronavirus may be 2019-nCoV.

Coronaviruses (CoVs), are enveloped positive-sense RNA viruses, which are surrounded by crown-shaped, club-like spikes projection on the outer surface. Coronaviruses' spike proteins are glycoproteins that are embedded over the viral envelope. This spike protein attaches to specific cellular receptors and initiates structural changes of spike protein, and causes penetration of cell membranes, which results in the release of the viral nucleocapsid into the cell. These spike proteins determine host trophism. Coronaviruses have a large RNA genome, ranging in size from 26 to 32 kilobases and capable of obtaining distinct ways of replication. Like other RNA viruses, coronaviruses under-go replication of the genome and transcription of mRNAs upon infection. Coronavirus infection in a subject can result in significant and long-term damage of the lungs, leading to possibly severe respiratory issues.

As used herein “2019-nCoV” is a betacoronavirus (Beta-CoV or β-CoV). In particular, 2019-nCoV is a Beta-CoV of lineage B. 2019-nCoV may also be known as SARS-CoV-2 or 2019 novel coronavirus. Betacoronaviruses are one of four genera of coronaviruses and are enveloped, positive-sense, single-stranded RNA viruses of zoonotic origin. Betacoronaviruses mainly infect bats, but they also infect other species like humans, camels, and rabbits. 2019-nCoV may be transferable between animals, such as between humans. As used herein “viral transmission” is the process by which viruses spread between host subjects. Transmission occurs from person to person by direct or indirect contact or exposure. Examples of direct contact include, but are not limited to, the exchange of body fluids between a subject infected with the virus and someone else. Indirect contact includes, but is not limited to, exposure to bodily fluid droplets produced by a subject infected by the virus during coughing and/or sneezing. Beta-CoVs may induce fever and respiratory symptoms in humans. The overall structure of β-CoV genome contains an ORF1ab replicase polyprotein (rep, pp1ab) preceding other elements. This polyprotein is cleaved into many nonstructural proteins. 2019-nCoV has a phenylalanine in the (F486) in the flexible loop of the receptor binding domain, flexible glycyl residues, and a four amino acid insertion at the boundary between the S1 and S2 subunits that results in the introduction of a furin cleavage site. The furin cleavage site may result in 2019-nCoV tissue tropism, increase transmissibility, and alter pathogenicity.

As used herein, “diagnosis of 2019-nCoV” comprises a positive test for 2019-nCoV and/or onset of 2019-nCoV symptoms, or combinations thereof. Symptoms of 2019-nCoV include, but are not limited to, one or more of the following symptoms: nasal congestion, sore throat, fever, body aches, exhaustion, dry cough, difficulty breathing, headache, shortness of breath, neuropathy-like symptoms, loss of taste, loss of smell, nausea, vomiting, diarrhea, or a combination thereof. Subjects at higher risk of developing complications may be immunocompromised (e.g., undergoing cancer treatment, bone marrow or organ transplantation, immune deficiencies, poorly controlled HIV or AIDS, prolonged use of corticosteroids or immune weakening medications), have an underlying medical condition (e.g., diabetes, renal failure, liver disease), are pregnant, are at least 65 years of age, have a chronic lung disease, have a heart disease, or combinations thereof.

Methods of treatment may include any number of modes of administering a disclosed composition. Modes of administration may include aerosols (e.g., via a nebulizer, inhaler, vaporizer, aerosolizer, and the like), tablets, pills, dragees, hard and soft gel capsules, granules, pellets, aqueous, lipid, oily or other solutions, emulsions such as oil-in-water emulsions, liposomes, aqueous or oily suspensions, syrups, elixirs, solid emulsions, solid dispersions or dispersible powders. For the preparation of pharmaceutical compositions for oral administration, the agent may be admixed with commonly known and used adjuvants and excipients such as for example, gum arabic, talcum, starch, sugars (such as, e.g., mannitol, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e.g., ethereal oils), solubility enhancers (e.g., benzyl benzoate or benzyl alcohol) or bioavailability enhancers (e.g., Gelucire™). In the pharmaceutical composition, the agent may also be dispersed in a microparticle, e.g., a nanoparticulate composition.

For parenteral administration, the agent can be dissolved or suspended in a physiologically acceptable diluent, such as, e.g., water, buffer, oils with or without solubilizers, surface-active agents, dispersants, or emulsifiers. As oils for example and without limitation, olive oil, peanut oil, cottonseed oil, soybean oil, castor oil and sesame oil may be used. More generally spoken, for parenteral administration, the agent can be in the form of an aqueous, lipid, oily or other kind of solution or suspension or even administered in the form of liposomes or nano-suspensions.

The term “parenterally,” as used herein, refers to modes of administration which include intravenous, inhalation, intramuscular, intraperitoneal, intrasternal, subcutaneous and intraarticular injection and infusion. In one non-limiting example, the AF composition can be administered by inhalation. In another non-limiting example, the AF composition can be administered intravenously.

### Kits

In one aspect, the disclosure provides kits comprising at least one disclosed AF or components thereof or a pharmaceutically acceptable salt thereof, and one or more of: a pharmaceutically acceptable carrier and an active agent.

In some embodiments, the at least one disclosed AF and components thereof and the at least one agent are co-formulated. In some embodiments, the at least one disclosed AF and components thereof and the at least one agent are co-packaged. The kits can also comprise AF and components thereof and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

The disclosed kits can be employed in connection with disclosed methods of use.

The kits may include information, instructions, or both that use of the kit will provide treatment for medical conditions in mammals (particularly humans). The information and instructions may be in the form of words, pictures, or both, and the like. In addition or in the alternative, the kit may include the AF and components thereof, a composition, or both; and information, instructions, or both, regarding methods of application of AF and components thereof, or of composition, preferably with the benefit of treating or preventing medical conditions in mammals (e.g., humans).

The AF and components thereof and processes of the disclosure will be better understood by reference to the following examples, which are intended as an illustration of and not a limitation upon the scope of the disclosure.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A method for treating lung disease in a subject in need thereof, the method comprising administering to the subject a therapeutically effective amount of a composition comprising a processed amniotic fluid having a therapeutically effective amount of protein, wherein the composition is substantially free of lanugo, vernix, and cells harvested with amniotic fluid.
Clause 2. The method of clause 1, wherein the protein comprises anti-inflammatory, anti-microbial, and/or immunomodulatory proteins.
Clause 3. The method of clauses 1 or 2, wherein the lung disease comprises inflammation of the lungs due to asthma, bronchitis, chronic obstructive pulmonary disease (COPD), pneumonia, restrictive lung disease, bronchiectasis, pulmonary fibrosis, sarcoidosis, allergies, smoking, emphysema, acute respiratory distress syndrome (ARDS), interstitial lung disease (ILD), pneumoconiosis, lung cancer, or a combination thereof.
Clause 4. The method of any one of the preceding clauses, wherein the lung disease involves one or more of alveoli, trachea, interstitium, pluera, bronchi, and bronchioles.
Clause 5. The method of any one of the preceding clauses, wherein administration of the composition results in a reduction in lung inflammation or improves lung function.
Clause 6. The method of any one of the preceding clauses, wherein the lung disease is caused by a bacterial infection or a viral infection.
Clause 7. The method of clause 6, wherein the viral infection is caused by an influenza virus, a respiratory syncytial virus, an adenovirus, a Varicella zoster virus, or a coronavirus.
Clause 8. The method of clause 7, wherein the coronavirus comprises Severe Acute Respiratory Syndrome (SARS-CoV), Middle East Respiratory Syndrome (MERS-CoV), COVID-19 (2019-nCoV, SARS-CoV-2), 229E, NL63, OC43, or HKU1.
Clause 9. The method of clause 6, wherein the bacterial infection is caused by Streptococcus pneumoniae, Mycobacterium tuberculosis, Bordetella pertussis, Haemophilus influenzae, Moraxella catarrhalis, Pseudomonas aeruginosa, Stenotrophomonas maltophila, Staphylococcus aureus, Streptococcus pyogenes, Neisseria meningitidis, Klebsiella pneumoniae, or Nontuberculosis Mycobacterium.
Clause 10. The method of any one of the preceding clauses, wherein the symptoms of the lung disease include one or more of shortness of breath, wheezing, coughing, yellow mucosa, green mucosa, blood-tinged mucosa, chest pain, breathlessness, rapid breathing, hypoxia, inflammation of the lung tissue, or a decrease in blood pressure.
Clause 11. The method of any one of the preceding clauses, wherein the lung disease is acute or chronic.
Clause 12. The method of any one of the preceding clauses, wherein the composition is administrated to the subject in need thereof via one or more administration protocols comprising, intravenous injection or inhalation.
Clause 13. The method of any one of the preceding clauses, wherein the composition comprises a therapeutically effective amount of hyaluronic acid (HA).
Clause 14. The method of any one of the preceding clauses, wherein the therapeutically effective amount of protein is from about 0.15 mg/mL to about 10 mg/mL.
Clause 15. The method of any one of the preceding clauses, wherein the composition is lyophilized.
Clause 16. The method of any one of the preceding clauses, wherein the composition has an optical density of less than 0.20 when exposed to electromagnetic radiation at a wavelength of 590 nm in a liquid form.
Clause 17. The method of any one of the preceding clauses, wherein the composition has less than or equal to 10,000 particles per milliliter of particles having a particle size of 10 microns or greater.
Clause 18. The method of any one of the preceding clauses, wherein the composition has less than or equal to 300 particles per milliliter of particles having a particle size of 25 microns or greater.
Clause 19. The method of any one of the preceding clauses, wherein the composition further comprises an active agent.
Clause 20. The method of clause 19, wherein the active agent is an anti-infective agent, an antibiotic, an anti-tumor agent, an anti-inflammatory agent, a pain-controlling agent, an anti-rheumatic agent, a bisphosphonate, a supplementary growth factor, a supplementary cytokine, an amino acid, a protein, a vaccine, a hormone, a vitamin, a phytoestrogen, fluoride, or combinations thereof.
Clause 21. The method of any one of the preceding clauses, wherein the composition is administered via inhalation and/or intravenous injection.
Clause 22. The method any one of the preceding clauses, wherein the composition is administered one or more times per day.
Clause 23. The method of any one of the preceding clauses, wherein the subject has been diagnosed with COVID-19.
Clause 24. The method of any one of the preceding clauses, wherein the composition is aerosolized and administered via a nebulizer.
Clause 25. The method of any one of the preceding clauses, wherein the subject receives at least 1 mL of the composition via inhalation every 24 hours.
Clause 26. The method of any one of the preceding clauses, wherein the subject further receives at least 1 mL of the composition intravenously every 24 hours.
Clause 27. Use of a therapeutically effective amount of a processed amniotic fluid having a therapeutically effective amount of protein, wherein the composition is substantially free of lanugo, vernix, and cells harvested with amniotic fluid for treating lung disease in a subject in need thereof.

### REFERENCES

1. Coronavirus 2019 (COVID-19): Pregnancy and breastfeeding [www.cdc.gov/coronavirus/2019-ncov/prepare/pregnancy-breastfeeding.html?CDC_AA_refVal=https%3A%2F%2Fwww.cdc.gov%2Fcoronavirus%2F2019-ncov%2Fspecific-groups%2Fpregnancy-faq.html]
2. Chen et al., “Clinical characteristics and intrauterine vertical transmission potential of COVID-19 infection in nine pregnant women: a retrospective review of medical records,” Lancet 395: 809-815 (2020).
3. Hamzelou, “Coronavirus: what we know so far about risks to pregnancy and babies,” New Scientist 2020.
4. Khan et al., “Association of COVID-19 with pregnancy outcomes in health-care workers and general women,” Clin. Microbiol. Infect. 26: 788-790 (2020).
5. Tarca et al., “Amniotic fluid cell-free transcriptome: a glimpse into fetal development and placental cellular dynamics during normal pregnancy,” BMC Med. Genomics 13: 25 (2020).
6. Cargnoni et al., “Amniotic membrane patching promotes ischemic rat heart repair,” Cell Transplant. 8(10):1147-1159 (2009).
7. Kim and Choi, “Neovascularization in a mouse model via stem cells derived from human fetal amniotic membranes,” Heart Vessels 26(2): 196-205 (2011).
8. Mao et al., “Processed human amniotic fluid retains its antibacterial activity,” J. Transl. Med. 17: 68 (2019).
9. Marsh et al., “Anti-inflammatory properties of amniotic membrane patch following pericardiectomy for constrictive pericarditis,” J. Cardiothorac. Surg. 12(1): 6 (2017).
10. O'Brien et al., “Evaluating the effects of platelet-rich plasma and amniotic viscous fluid on inflammatory markers in a human coculture model for osteoarthritis,” Arthroscopy 5(8):
11. Tahmasebi et al., “Prevention by rat amniotic fluid of adhesions after laparatomy in a rat mode,”. Int. J. Surg. 10(1): 16-19 (2012).
12. Pierce et al., “Collection and characterization of amniotic fluid from scheduled C-section deliveries,” Cell Tissue Bank 17: 413-425 (2016).
13. Koob et al., “Biological properties of dehydrated human amnion/chorion composite graft: implications for chronic wound healing,” Int. Wound J. 10(5): 493-500 (2013).
14. Liu et al., “Update on amniotic membrane transplantation,” Expert Rev. Ophthalmol. 5(5): 645-661 (2010).
15. Mohammadi et al., “Effect of fresh human amniotic membrane dressing on graft take in patients with chronic burn wounds compared with conventional methods,” Burns 39(2): 349-353 (2013).
16. Tay et al., “The trinity of COVID-19: immunity, inflammation and intervention,” Nat. Rev. Immunol. 20: 363-374 (2020).
17. Stone et al., “Efficacy of tocilizumab in patients hospitalized with Covid-19,” N. Engl. J. Med. 383(24): 2333-2344 (2020).
18. Cavalcanti et al., “Hydroxychloroquine with or without azithromycin in mild-to-moderate Covid-19,” N. Engl. J. Med. 383(21): 2041-2052 (2020).
19. Madjid et al., “Potential effects of coronaviruses on the cardiovascular system: a review,” JAMA Cardiol. 5(7): 831-840 (2020).
20. Tse et al., “Pulmonary pathological features in coronavirus associated severe acute respiratory syndrome (SARS),” J. Clin. Pathol. 57(3): 260-265 (2004).
21. Tian et al., “Pulmonary pathology of early-phase 2019 novel coronavirus (COVID-19) pneumonia in two patients with lung cancer,” J. Thorac. Oncol. 15(5): 700-704 (2020).

### EXAMPLES

### Example 1

### Administration of Amniotic Fluid Composition to COVID-19 Patients in Critical and Non-Critical Conditions

To recruit and identify participants, physicians and research staff reviewed electronic medical records to identify patients in the hospital who have been diagnosed with COVID-19 and confirmed as positive for COVID-19.

Amniotic fluid (AF) was procured by the Cell Therapy and Regenerative Medicine (CellReGen) Group at the University of Utah under IRB 036454. Frozen or thawed product was delivered by this group to the relevant hospital unit/floor.

The following measurements were obtained after consent and enrollment, but prior to intervention: date of symptom onset, vitals (e.g., heart rate, blood pressure, oxygen saturation, respiratory rate, temperature), Glasgow Coma Scale score, arterial blood gas, respiratory support modality (e.g., nasal cannula, simple face mask, non-rebreather, non-invasive positive pressure ventilation [NIPPV], high flow nasal cannula, invasive mechanical ventilation) and settings, including oxygen administration (e.g., LPM or FiO₂), complete blood count (CBC) with differential, complete metabolic panel (CMP), inflammatory biomarkers (e.g., IL-6, LDH, D-Dimer, CRP).

Patients on the floor (e.g., cardiovascular medical unit (CVMU) or acute internal medicine-A (AIM-A) unit) received 3 mL (3 cc) of nebulized AF once every 24 hours for 5 days, and/or 1-10 mL (1-10 cc) of intravenous amniotic fluid once every 24 hours for 5 days. Patients in an intensive care unit (e.g., medical intensive care unit (MICU) or cardiovascular intensive care unit (CVICU) received 3 mL (3 cc) of nebulized amniotic fluid once every 24 hours for 5 days and/or 1-10 mL (1-10 cc) of intravenous amniotic fluid once every 24 hours for 5 days. Daily assessments were taken as described above for the pre-intervention assessment except for CBC and CMP assessments.

The AF coming from donors were screened for infectious diseases through a medical and social history questionnaire, medical record review, and testing of the donor's blood for viruses and infectious disease agents including HIV, Hepatitis B or C, and others.

Patients administered the AF composition were monitored for signs of an immune reaction to the nebulized and/or intravenous amniotic fluid. Treatment was provided if needed.

At ICU discharge, the pre-intervention assessment was repeated with the addition of viral load of COVID-19.

The following primary outcomes were measured: ventilator free days (only Group 1—ICU patients), days alive and off mechanical ventilation at day 60 (measured from hospital admission, day 60 after admission), duration of supplemental oxygen use (only Group 2—non-ICU patients), and duration from hospital admission until cessation of supplemental oxygen use (measured from hospital admission to day 60).

The following secondary outcomes were measured: all-cause mortality, survival at day 60 or hospital discharge (measured at day 60 or at hospital discharge, whichever comes first), systemic inflammation, and systemic inflammation at 5 days measured by serum IL-6 (measured at day 5 post enrollment).

Descriptive statistics, including mean (SD) and median (interquartile range), were used to assess patient characteristics, and clinical and biomarker outcomes. Comparisons were made stratified on ICU vs floor status at enrollment for variables. Categorical characteristics were compared using chi-square test or Fisher exact test. Continuous characteristics were compared using independent samples t-test or Wilcoxon-Mann-Whitney test. 95% Confidence intervals and p-values were reported from all comparisons. Statistical analyses were conducted in R and STATA, significance was assessed at the 0.05 level and all tests were two-tailed.

### Example 2

### Overview of Study Design

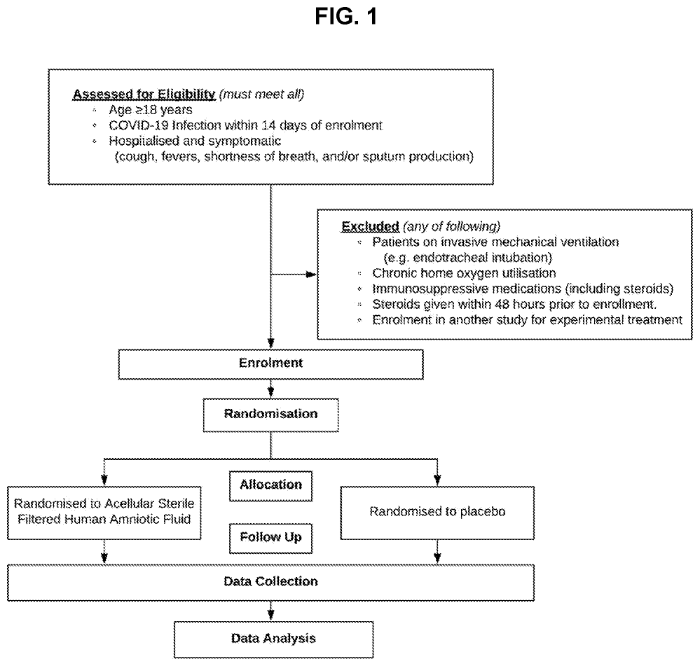

The trial began enrolment on 28 Oct. 2020 as a single-centre, prospective, randomised, double-blinded, placebo-controlled trial. Participants are recruited from hospitalized in patients with COVID-19. FIG. 1 shows the study screening and enrolment flow chart. The trial target enrolment was 60 patients. This study protocol followed the Standard Protocol Items: Recommendations for Interventional Trials, version 2013. The hypothesis was that daily intravenous administration of acellular sterile filtered hAF in hospitalized patients with symptomatic COVID-19 would decrease inflammatory biomarker CRP compared with placebo.

### Participant Selection and Recruiting Process

We screened hospitalised patients with confirmed COVID-19 infection among hospitalised patients. Ideal subjects are mild to moderately, but not severely, ill with COVID-19. Screening included an initial electronic medical record review by study coordinators at each site to identify patients with laboratory-confirmed COVID-19. Patient notes were screened for evidence of active symptoms as defined in inclusion criteria. Given the contagiousness of COVID-19, eligible patients, or their legally authorised representatives (LAR), will be contacted via phone for e-consent.

### Inclusion Criteria

Patients are eligible for trial enrolment if they fulfil all the criteria:
At least 18 years of age.
SARS-CoV-2 laboratory positive test, obtained within 14 days of enrolment.
Hospitalised and symptomatic (cough, fevers, shortness of breath and/or sputum production).
Has a room air pulse oximetry of ≤94% and requires supplemental oxygen therapy.
Patients of childbearing potential who agree to use acceptable methods of contraception for 90 days after last administration of study investigational product (IP).
Patients who are receiving standard of care therapies for COVID-19 that are not FDA approved are eligible for this study.
Patients must be able to consent to the study (eg, Glasgow Coma Scale score ≥14).
Patients are required to have controlled blood pressure <160/96 and have a pulse <110.

### Exclusion Criteria

Patients will be excluded if they fulfil any of the exclusion criteria:
Patients on invasive mechanical ventilation (eg, endotracheal intubation).
Patients on non-invasive positive pressure ventilation.
Patients on >12 L/min via non-rebreather or >80% oxygen via high flow nasal cannula.
Patients who, in the opinion of the investigator, have impending respiratory failure, defined as requiring rapidly escalating oxygen supplementation.
Chronic home oxygen utilization.
Home use of immunosuppressive medications (including steroids).
Women who are pregnant, breast feeding or become pregnant during the study.
Patients with a haemoglobin <90 g/L.
Patients diagnosed with stage 4 or 5 chronic kidney disease.
Patients diagnosed with class 3 or 4 congestive heart failure.
Patients with a left ventricular assist device.
Patients with thromboembolic phenomena.
Patients with type 2 and above heart block.
Patients with established positive bacterial blood cultures prior to enrolment.
Patients with ongoing pericardial effusion, or ascites.
Patients with clinically significant arrhythmia.
Patients with liver function tests (alanine aminotransferase or aspartate aminotransferase)>3× normal.
Patients with untreated HIV infection.
Patients diagnosed with end-stage organ disease.

### Ethics and Informed Consent

This trial was approved by the University of Utah Institutional Review Board (IRB_0013292), received approval from the US FDA for an IND (No 23369) and is registered on ClinicalTrials.gov. The trial registration adheres to all items from the WHO Trial Registration Data Set. The study protocol (Version: 10 Aug. 2020) is listed in the online supplemental file 1. Written informed consent is obtained by study staff from each enrolled patient. The consent form is available in the online supplemental file 1 and includes a discussion of the risks and benefits of participation, and the ability to withdraw at any time without consequence.

This study was published as Tonna et al., “Safety and feasibility of using acellular sterile filtered amniotic fluid as a treatment for patients with COVID-19: protocol for a randomised, double-blinded, placebo-controlled clinical trial,” BMJ Open (2021) 11: e045162. doi:10.1136/bmjopen-2020-045162, which is incorporated by reference in its entirety.

### Example 3

Early data in the COVID-19 pandemic suggested that vertical transmission from SARS CoV-2-infected women is uncommon and virus has not been detected in the amniotic fluid [1-4]. These observations raise the question of whether or not there is something intrinsically protective in the fluid. Indeed, human amniotic products have a broad immune mediating profile [5]. Human amniotic membrane (hAM) and human amniotic fluid (hAF) have been shown to reduce inflammation, have antimicrobial properties, and confer a low risk of immunogenicity [6-11]. Purified hAF is non-antigenic solution devoid of any cellular products (i.e., it is not to be confused with umbilical cord-derived, AF-derived stem cell products, or AF embolism) that nature developed [5, 12]. Hence, amniotic products make ideal biocompatible scaffolds for the treatment of diverse conditions, including but not limited to intractable epithelial defects, burns, diabetic/peripheral vascular ulcers, partial limbal cell deficiencies, peripheral nerve regeneration, tendon repair, and Stevens-Johnson syndrome [13-15].

Our group has developed experience with the clinical use of amniotic products and is currently using hAM as a tissue allograft in burn patients, for digital ulcers in scleroderma patients, as a nerve wrap to protect nerves from adhesions post-surgery, and as a trachea-stenosis-tracheal stent covering. Additionally, our burn unit has successfully injected purified hAF into more than 500 burn patient wounds to augment graft survival. hAF is also currently being used experimentally under IND approval to treat ocular graft versus host disease and ocular PRK. Given the use of amniotic products for the treatment of inflammation in other conditions, our group proposed an experimental and innovative use of hAF as its impact on cardiopulmonary failure has not been previously investigated. In light of the profound local and systemic inflammation associated with COVID-19 [16], it is our global hypothesis that hAF could significantly mitigate the progression of disease. An expedited protocol was approved by the University of Utah Institutional Review Board to study the influence of hAF in 10 patients with confirmed COVID-19. Herein, we report the results of this case series.

Purified hAF is a cell-free, non-antigenic solution (not to be confused with umbilical cord-derived or AF-derived stem cell products) that is processed and manufactured at the University of Utah for clinical use [8, 12].

The initial trial design identified two cohorts of hospitalized, symptomatic, and laboratory verified SARS CoV-2 patients: (1) mechanically ventilated patients were administered hAF both intravenously (3 cc) and nebulized (3 cc) for 5 days; and (2) non-mechanically ventilated patients were administered only nebulized (3 cc) hAF. After the first 4 patients, concerns with utilizing aerosolized therapies in COVID-19 patients required a temporary hold and subsequent re-design. Thereafter (the last 6 patients), all received a higher dose of intravenous hAF (10 cc) for 5 consecutive days. Patients were eligible for this study if there were over the age of 18, had a SARS CoV-2 laboratory positive test obtained within 14 days of enrollment, were hospitalized, and symptomatic (e.g., cough, fevers, shortness of breath, sputum production). There were no exclusion criteria for the case series. Furthermore, to the best of our knowledge, there are no absolute contraindication for the clinical use of our purified hAF. Participants were enrolled over the span of seven weeks between late March and early May of 2020.

The demographics (Table 1) of the total 10 patient cohort included the following: 40% female; average age of 51.9 years old (range 24-76); five White, two American Indian/Alaska Native, two Hispanic, and one as unknown/other. Average weight was 91 kg (range 42-140). Eight of the 10 participants had underlying comorbid conditions, of which the most common were diabetes (n=6) and hypertension (n=5). As this is a small case series, pertinent information is provided in a patient-by-patient manner (Table 1). Of the 10 patients enrolled, seven were admitted directly to the intensive care unit (ICU). All ten patients required oxygen support during their hospital admission. Three patients required a nasal cannula or simple mask as their highest level of support, while two were placed on a high flow nasal cannula as their highest support. Five patients required mechanical ventilation. One patient (#1) was enrolled on high-level ventilatory support (100% FiO₂, 18 mm Hg PEEP) in the prone position and ultimately required 41 days of veno-veno extracorporeal membrane oxygenation (initiated after 5th day of hAF treatment) as well as tracheostomy, which was removed prior to discharge. Another patient (#6) required a tracheostomy and was discharged with this still in place, although he was no longer on mechanical ventilation. Of the nine patients who have been discharged alive, 7 required home oxygen use.

TABLE 1
Demographics and Medical History

| Variable | n |
|---|---|
| Age (mean, range) | 52, 24-76 |
| Female sex | 4 |
| Weight (kg; mean, range) | 91 (42-140) |
| Race/Ethnicity | |
| White | 5 |
| American Indian/Alaskan Native | 2 |
| Hispanic | 2 |
| Unknown/other | 1 |
| Medical History | |
| Anemia | 1 |
| Coronary artery disease | 1 |
| Diabetes | 6 |
| Elevated liver enzymes | 1 |
| Heart murmur | 1 |
| Hyperlipidemia | 2 |
| Hypertension | 5 |
| Liver disease | 1 |
| Mitral valve disease | 1 |
| Respiratory disease (e.g., asthma) | 2 |
| No past medical history | 2 |

Results of primary outcome measures included hospital length of stay, ICU length of stay, ventilator-free days, and supplemental oxygen needs at the time of discharge (Table 2). The 4 patients who were mechanically ventilated and ultimately discharged alive average 13.5 ventilator-free days (range 5-28). With regard to supplemental oxygen use at the time of discharge, 2 patients were entirely on room air, 6 were requiring continuous oxygen, and 1 required intermittent oxygen use. Average hospital length of stay (n=10) was 20.4 days (range 5-56 days). Nine of 10 patients were admitted to the ICU at some point during their hospitalization. Average ICU length of stay for surviving patients (n=8) was 17.38 days (range 2-56 days). For this subgroup of 8 patients, the average ICU-free days/length of stay on the medical floor was 5.9 days (range 1-19 days).

TABLE 2
Outcomes of 10 Enrolled Patients

| | | Init. | Max. | | | | | | |
|---|---|---|---|---|---|---|---|---|---|
| No | Admit | O₂ | O₂ | Co-Rx | CRP | IL-6 | D-dimer | LDH | Discharge |
| 1 | ICU | ETT | ECMO/ | HCQ | +30% | +38% | +138% | −27% | Alive |
| | | | Trach | | | | | | |
| 2 | Floor | NC | NC | HCQ | N/A | N/A | N/A | −28% | Alive |
| 3 | Floor | NC | ETT | HCQ, | N/A | N/A | N/A | −20% | Deceased |
| | | | | Rem | | | | | |
| 4 | ICU | NC | NC | HCQ | N/A | N/A | N/A | N/A | Alive |
| 5 | ICU | ETT | ETT | HCQ | −84% | −60% | −21% | −22% | Alive |
| 6 | ICU | ETT | Trach | HCQ- | −91% | 0 | −42% | N/A | Alive |
| | | | | AZ | | | | | |
| 7 | ICU | HFNC | HFNC | HCQ | +46% | +109% | +818% | +109% | Alive |
| 8 | ICU | NC | HFNC | AZ | −87% | 0 | 0 | −21% | Alive |
| 9 | ICU | NC | Simple | HCQ | −80% | −20% | −28% | +109% | Alive |
| | | | mask | | | | | | |
| 10 | ICU | HFNC | ETT | HCQ | +41% | +412% | +33% | −30% | Alive |

Of note, protocol change (10 cc hAF I.V.) for patients 5-10.
N/A not available, ECMO extracorporeal membrane oxygenation, ETT endotracheal intubation, HFNC high flow nasal cannula, ICU intensive care unit, MACE major adverse cardiac event, NC nasal cannula, O2 oxygen, Trach tracheostomy, Co-Rx Co-treatment, HCQ hydroxychloroquine, AZ azithromycin; Rem remdesivir.
Biomarker are represented as a percent change between study enrollment and termination of hAF (5 days) with focus on the last 6 patients under current protocol.
CRP: C-reactive protein;
IL-6: interleukin-6;
LDH: lactate dehydrogenase

Biomarkers were evaluated before and after treatment with hAF (Table 1, Table 3). Because of the small sample size and some missing data, it is difficult to make any broad statements concerning the influence of hAF on these biomarkers. That stated, in the latter cohort that received 10 cc hAF intravenously, a mean reduction in C-reactive protein by 38% was observed. Particularly notable is an apparent decrease in markers in those patients (4/6) that had improved clinical profiles. Conversely, two of the six patients saw increases in these inflammatory biomarkers—one had a prolonged hospitalization and the other was discharged to a rehabilitation hospital. One patient (#3) died on hospital day 8. She had multiple comorbidities including morbid obesity (BMI 55 kg/m2) and was extremely ill (maximal ventilatory support) on admission and had a noticeable increase in her biomarkers. Outside of the above-mentioned issue related to aerosolized therapy, where theoretic concerns about safety to the provider delivering the nebulized hAF were never observed, there were no reported safety concerns.

TABLE 3
Raw biomarker data before and after
therapy for each individual patient

| | CRP (mg/dl) | | IL-6 (pg/ml) | | D-dimer (μg/ml) | | LDH (U/L) | |
|---|---|---|---|---|---|---|---|---|
| Patient | Pre | Post | Pre | Post | Pre | Post | Pre | Post |
| 1 | 23.1 | 21.4 | 57 | 248 | 2.1 | 14.3 | 530 | 387 |
| 2 | N/A | N/A | N/A | N/A | N/A | N/A | 313 | 317 |
| 3 | N/A | N/A | N/A | N/A | N/A | N/A | 439 | 336 |
| 4 | N/A | N/A | N/A | N/A | N/A | N/A | N/A | 249 |
| 5 | 8.2 | 1.3 | 10 | 4 | 1.9 | 1.5 | 395 | 307 |
| 6 | 3.7 | 0.3 | 4 | 4 | 1.9 | 1.1 | N/A | N/A |
| 7 | 15.6 | 22.8 | 22 | 46 | 1.6 | 14.7 | 539 | 679 |
| 8 | 7.8 | 1 | 4 | 4 | 1.3 | 1.3 | 400 | 315 |
| 9 | 12.4 | 5.9 | 5 | 4 | 0.7 | 0.6 | 241 | 261 |
| 10 | 20.4 | 28.8 | 65 | 333 | 2.7 | 3.6 | 551 | 384 |

N/A: not available; CRP: C-reactive protein; IL-6: interleukin-6; LDH: lactate dehydrogenase.

The body of knowledge related to the pathophysiology and therapeutics of COVID is rapidly evolving. Our understanding of the relative contributions of innate and adaptive immune responses is similarly advancing. Indeed, many early observations have been modified as the nuances of different patient populations and presentations exist. Some patients, for example, demonstrate a syndrome consistent with cytokine storm, with a very aggressive inflammatory response. These patients might benefit from blockade of classic cytokine pathways (i.e. IL-6 antagonists). Other patients have a more subtle, perhaps less florid inflammatory response, and might not benefit as much from robust immune-modulation.

One of the attractive features of hAF is its diverse “soup” of ingredients. Expression profiling of 68 term gestation patients demonstrated that the cell-free AF transcriptome contains over 64,000 genes [5, 12]. At our center, we performed a cytokine array on AF from 17 patients with normal term pregnancy. Over 300 proteins (out of the 400 on the array) were present in AF with the majority associated with host defense and angiogenesis [12]. Biology has long taught us that blocking or augmenting a single pathway is confounded by naturally derived redundancy and alternative feedback loops. Indeed, while IL-6 is found to be upregulated in COVID patients, treatment with a singular antagonist does not necessarily change the course of the disease [17]. While some therapeutics will try to combine various mechanistic pathways [18], hAF is nature's combination of not just one or two proteins, but rather thousands of proteins which have been mixed together and survived thousands of years.

Outside of the aforementioned studies surrounding wound healing, there is scant data with regards to the utilization of hAF for cardiopulmonary disease. Many COVID-19 patients, in addition to succumbing to acute hypoxic respiratory failure, are plagued with cardiac manifestations including myocarditis, accelerated heart failure, and arrhythmias [19]. Lung pathology specimens from the SARS-1 epidemic in 2005 demonstrated diffuse alveolar damage with extensive fibrosis [20]. Likewise, lung pathology in COVID-19 patients undergoing lung resection for lung cancer (i.e., non-autopsy pathology) revealed edema and patchy, inflammatory cellular infiltrates, particularly with macrophages [21]. Summarily, COVID-19 can cause an aggressive inflammatory and fibrotic response in both the heart and lung. This suggests that hAF, by mitigating inflammation and decreasing fibrosis, could impact the natural history of COVID-19 infected patients and provides the foundational platform for our investigation of the potential impact of systemic administration of hAF in COVID-19 patients.

This case series has a number of limitations including the small patient cohort (thereby limiting inferential and comparative statistics), mixed patient population including those in extremis, mixed delivery protocol between the first 4 and the last 6 patient, and concomitant medical and experimental therapies (for example, hydroxychloroquine—see Table 1). In addition, this case series was performed in the initial phases of the pandemic and some of the biomarker studies in the first 4 patients were not available or collected within the time frame set forth by protocol. Despite these significant caveats, the observations in these patients suggest that hAF can be used safely in this population and provides a potential signal of a favorable biologic influence. As such, this report provides some data to support the conceptual rationale for further investigation. This experience has propelled our application and subsequent approval from the FDA for IND status (#23,369) and has informed the design and recent implementation of a larger-scale, randomized clinical trial to evaluate the efficacy and novel utilization of human amniotic fluid as therapy for COVID-19.

This study was published as Selzman et al., “A pilot trial of human amniotic fluid for the treatment of COVID [symbol] 19,” BMC Res. Notes (2021) 14: 32 doi.org/10.1186/s13104-021-05443-9, which is incorporated by reference in its entirety.

1. A method for treating lung disease in a subject in need thereof, the method comprising administering to the subject a therapeutically effective amount of a composition comprising a processed amniotic fluid having a therapeutically effective amount of protein, wherein the composition is substantially free of lanugo, vernix, and cells harvested with amniotic fluid.

2. The method of claim 1, wherein the protein comprises anti-inflammatory, anti-microbial, and/or immunomodulatory proteins.

3. The method of claim 1, wherein the lung disease comprises inflammation of the lungs due to asthma, bronchitis, chronic obstructive pulmonary disease (COPD), pneumonia, restrictive lung disease, bronchiectasis, pulmonary fibrosis, sarcoidosis, allergies, smoking, emphysema, acute respiratory distress syndrome (ARDS), interstitial lung disease (ILD), pneumoconiosis, lung cancer, or a combination thereof.

4. The method of claim 1, wherein the lung disease involves one or more of alveoli, trachea, interstitium, pluera, bronchi, and bronchioles.

5. The method of claim 1, wherein administration of the composition results in a reduction in lung inflammation or improves lung function.

6. The method of claim 1, wherein the lung disease is caused by a bacterial infection or a viral infection.

7. The method of claim 6, wherein the viral infection is caused by an influenza virus, a respiratory syncytial virus, an adenovirus, a Varicella zoster virus, or a coronavirus.

8. The method of claim 7, wherein the coronavirus comprises Severe Acute Respiratory Syndrome (SARS-CoV), Middle East Respiratory Syndrome (MERS-CoV), COVID-19 (2019-nCoV, SARS-CoV-2), 229E, NL63, OC43, or HKU1.

9. The method of claim 6, wherein the bacterial infection is caused by Streptococcus pneumoniae, Mycobacterium tuberculosis, Bordetella pertussis, Haemophilus influenzae, Moraxella catarrhalis, Pseudomonas aeruginosa, Stenotrophomonas maltophila, Staphylococcus aureus, Streptococcus pyogenes, Neisseria meningitidis, Klebsiella pneumoniae, or Nontuberculosis Mycobacterium.

10. The method of claim 1, wherein the symptoms of the lung disease include one or more of shortness of breath, wheezing, coughing, yellow mucosa, green mucosa, blood-tinged mucosa, chest pain, breathlessness, rapid breathing, hypoxia, inflammation of the lung tissue, or a decrease in blood pressure.

11. The method of claim 1, wherein the lung disease is acute or chronic.

12. The method of claim 1, wherein the composition is administrated to the subject in need thereof via one or more administration protocols comprising, intravenous injection or inhalation.

13. The method of claim 1, wherein the composition comprises a therapeutically effective amount of hyaluronic acid (HA).

14. The method of claim 1, wherein the therapeutically effective amount of protein is from about 0.15 mg/mL to about 10 mg/mL.

15. The method of claim 1, wherein the composition is lyophilized.

16. The method of claim 1, wherein the composition has an optical density of less than 0.20 when exposed to electromagnetic radiation at a wavelength of 590 nm in a liquid form.

17. The method of claim 1, wherein the composition has less than or equal to 10,000 particles per milliliter of particles having a particle size of 10 microns or greater.

18. The method of claim 1, wherein the composition has less than or equal to 300 particles per milliliter of particles having a particle size of 25 microns or greater.

19. The method of claim 1, wherein the composition further comprises an active agent.

20. The method of claim 19, wherein the active agent is an anti-infective agent, an antibiotic, an anti-tumor agent, an anti-inflammatory agent, a pain-controlling agent, an anti-rheumatic agent, a bisphosphonate, a supplementary growth factor, a supplementary cytokine, an amino acid, a protein, a vaccine, a hormone, a vitamin, a phytoestrogen, fluoride, or combinations thereof.

21. The method of claim 1, wherein the composition is administered via inhalation and/or intravenous injection.

22. The method of claim 1, wherein the composition is administered one or more times per day.

23. The method of claim 1, wherein the subject has been diagnosed with COVID-19.

24. The method of claim 1, wherein the composition is aerosolized and administered via a nebulizer.

25. The method of claim 1, wherein the subject receives at least 1 mL of the composition via inhalation every 24 hours.

26. The method of claim 1, wherein the subject further receives at least 1 mL of the composition intravenously every 24 hours.

27. (canceled)
